(19) United States
(12) Patent Application Publication
(10) Pub. No.: US 2006/0270620 A1
(43) Pub. Date: Nov. 30, 2006

(54) Method of Enhancing Therapeutic Effect of Nucleic Acids

(75) Inventors: Loree Heller, Tampa, FL (US); Richard Gilbert, Tampa, FL (US); Mark J. Jaroszeski, Tampa, FL (US); Richard Heller, Tampa, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 11/161,665

(22) Filed: Aug. 11, 2005

Related U.S. Application Data
(63) Continuation-in-part of application No. 10/064,512, filed on Jul. 23, 2002, now abandoned.

(51) Int. Cl. A61K 48/00; A61N 1/00

(52) U.S. Cl. 514/44; 607/2

(57) ABSTRACT

The present invention is a method of eliciting an antitumor effect in vivo comprising the steps of identifying a species representative of a treatment subject, identifying at least one non-coding nucleic acid sequence, introducing the at least one nucleic acid to at least one tumor in the treatment subject and applying an energy source to the at least one tumor. The energy source may comprise, but is not limited to, electrical, sonic, photonic, and microwave output.

### CROSS REFERENCE TO RELATED APPLICATIONS

This application is a continuation-in-part of U.S. patent application Ser. No. 10/064,512 filed Jul. 23, 2002, which claims priority from U.S. Provisional Patent Application No. 60/307,523 filed Jul. 24, 2001.

### FIELD OF INVENTION

This invention relates to the delivery of nucleic acids into cells, and more particularly to a method of making nucleic acid that is non-coding therapeutically effective by electromanipulation.

### BACKGROUND OF INVENTION

The use of nucleic acids as therapeutic molecules has long been studied for the treatment of cancer and metabolic disease in humans as well as other animals. Many types of nucleic acids have been investigated including deoxyribonucleic acid (DNA) and ribonucleic acid (RNA) in both their single and double stranded forms. In addition, nucleic acids that have been modified from their normally occurring forms have also been investigated as potentially therapeutic. Different sequences of the nucleotides that comprise nucleic acids have also been investigated. Sequences that code for a transcription or translation product or interfere with transcription or translation of a product that are potentially therapeutic are normally investigated for use in therapies. These, for example, include nucleotide sequences that code for cytokines, antigens, secreted cellular products, and antisense sequences. Typically, human sequences that code for the therapeutic molecule are used for therapy in humans. However, the present invention stems from the counterintuitive discovery that DNA that does not code for a translation product can result in cellular effects. These effects were observed to be tumor regression after the DNA sequence was delivered to tumor cells in vivo using electricity.

Nucleic acids must be inside a cell in order a cellular effect to occur. One method for delivering DNA to cells is to use electric fields to mediate the internalization of nucleic acids by cells. Scientific research has led to the current understanding that exposure of cells to intense electric fields for brief periods of time temporarily destabilized membranes; however, there may be other effects that have not yet been elucidated. This effect has been described as a dielectric breakdown due to an induced transmembrane potential, and was termed “electroporation”, or “electropermeabilization”, because it was observed that molecules that do not normally pass through the membrane gain intracellular access after the cells were treated with electric fields. The porated state was noted to be temporary. Typically, cells remain in a destabilized state on the order of minutes after electrical treatment ceases.

The physical nature of electroporation makes it universally applicable. A variety of procedures utilize this type of treatment, which gives temporary access to the cytosol. These include production of monoclonal antibodies and genetic transformation. In addition, dyes and fluorescent molecules have been used to investigate the phenomenon of electroporation. A notable example of loading molecules into cells in vivo is electrochemotherapy. The procedure utilizes a drug combined with electric pulses as a means for loading tumor cells with an anticancer drug and has been performed in a number of animal models and in clinical trials by the present inventors.

The loading of molecules by electroporation in vivo is typically, but not necessarily, carried out by first exposing the cells or tissue of interest to the molecule to be loaded. This is accomplished by placing the molecules of interest into the extracellular space by injection, jet injection or other means. The cells or tissue are then exposed to electric fields by administering one or more direct current pulses. Pulses are normally applied using an electrical generator and electrodes that contact the cells/tissue. Electrical treatment is conducted in a manner that results in a temporary membrane destabilization with minimal cytotoxicity. The intensity of electrical treatment is described by the magnitude of the applied electric field. This field is defined as the voltage applied to the electrodes divided by the distance between the electrodes. Electric field strengths ranging from 100 to 5000 V/cm have been used and are specific to the cells or tissue under investigation. Pulses are usually rectangular in shape; however, exponentially decaying pulses have also been used. The duration of each pulse is called pulse width. Molecule loading has been performed with pulse widths ranging from microseconds to milliseconds. Single or multiple pulses may be delivered. Typically, multiple pulses are utilized during electrical treatment.

There are other energy based systems for delivering molecules in vivo. The use of acoustic energy has been used to, in a manner similar to electric fields, to facilitate the uptake of molecules by cells in vivo. Other energy sources such as light and microwave energy have the same membrane disruptive effect.

It is therefore an object of the present invention to effect long-term or permanent tumor regression by in vivo application of energy to tumor containing exogenous non-coding nucleic acids.

It is, therefore, to the effective resolution of the aforementioned problems and shortcomings of the prior art that the present invention is directed.

However, in view of the prior art in at the time the present invention was made, it was not obvious to those of ordinary skill in the pertinent art how the identified needs could be fulfilled.

### SUMMARY OF INVENTION

The present invention is a method of eliciting an antitumor effect in vivo comprising the steps of identifying a species representative of a treatment subject, identifying at least one non-coding nucleic acid sequence, introducing the at least one non-coding nucleic acid to at least one tumor in the treatment subject and applying an energy source to the at least one nucleic acid. The energy source may comprise, but is not limited to, electrical, sonic, photonic, and microwave output. Preferably, the energy source is adapted to make permeable at least one cell in the at least one tumor by an applied electrical strength between 100 to 5,000 volts per centimeter emitted by a plurality of electrical pulses. At least one nucleic acid is introduced to at least one tumor in the treatment subject by injecting or jet injecting the nucleic acid into extracellular space coincident to at least one tumor.

### BRIEF DESCRIPTION OF THE DRAWINGS

For a fuller understanding of the nature and objects of the invention, reference should be made to the following detailed description, taken in connection with the accompanying drawings, in which:

FIG. 1 is a diagrammatic view of the method according to the invention.

FIG. 2 shows the effect of three intratumor electroporation deliveries of plasmid DNA on tumor volume.

FIG. 3 shows tumor regression data confirming eukaryotic coding sequences are not necessary for the antitumor effect.

FIG. 4 illustrates the result of an exemplary embodiment of the present invention in which electrically mediated delivery of CpG motif oligonucleotide, but not control oligonucleotide, induces tumor regression

FIG. 5 shows a histological analysis of paraffin-embedded sections by hematoxylin and eosin staining 24 hours after treatment.

FIG. 6 shows histological analysis of paraffin-embedded sections by hematoxylin and eosin and TUNEL staining 24 hours after delivery of 100 μg plasmid DNA (VR1255) using electroporation.

### DETAILED DESCRIPTION OF THE PREFERRED EMBODIMENT

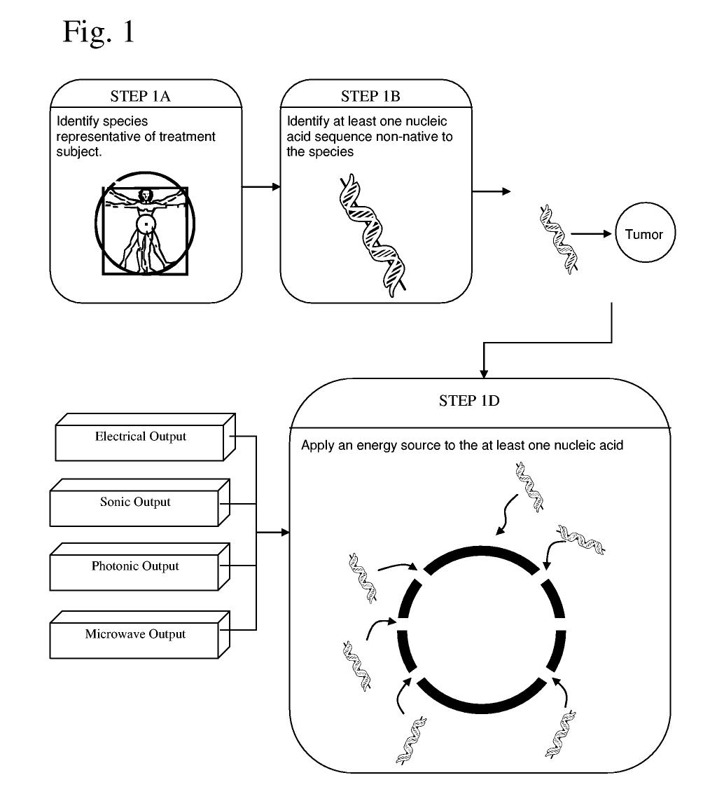

FIG. 1 illustrates the general method according to the invention including identifying a species representative of a treatment subject (1A), identifying at least one non-coding nucleic acid sequence (1B), introducing the at least one nucleic acid to at least one tumor in the treatment subject (1C) and applying an energy source to the at least one nucleic acid (1D). The energy source may include, individually or in combination, electrical, sonic, photonic or microwave output.

In a specific exemplary embodiment of the present invention, a plasmid (pUC18), constructed of DNA, was propagated in the bacteria E. coli and purified using a Qiagen plasmid preparation kit (Qiagen, Valencia, Calif.). The plasmid PUC18 is a traditional DNA sequence. There were no species specific/mammalian DNA sequences in the plasmid that coded for a protein. Also, the plasmid did not contain a promoter. There was no mammalian DNA in the plasmid; it is what is known as an empty plasmid. This plasmid DNA sequence was used below.

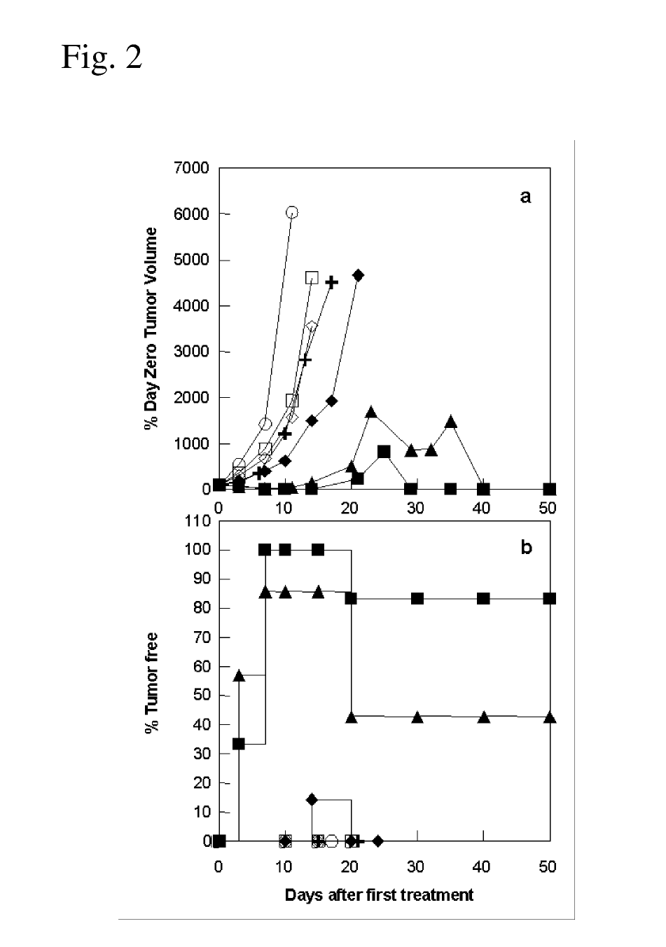

With reference to FIG. 2, the effect of three intratumor electroporation deliveries of plasmid DNA on tumor volume are illustrated. In accordance with this exemplary embodiment, after tumors grew to a mean diameter of 40 mm (day 0), tumors were treated with VR1255, a plasmid encoding firefly luciferase, a non-therapeutic gene, and electroporation on days 0, 3, and 7. Tumors were then measured twice weekly using a digital caliper. Tumor volume was calculated by the standard formula v=ab2π/6, where a is the longest diameter, and b is the next longest diameter perpendicular to a. In the case of continued tumor growth or tumor recurrence, the animal was considered incurable and humanely euthanized when the tumor volume reached 1000 mm3. Each individual tumor volume was normalized to its volume on day 0, the first day of treatment. (a)Tumor volumes and (b) tumor free animals after delivery of plasmid DNA (VR1255) with electroporation. Wherein an empty circle indicates no treatment; a plus sign indicates injection of 100 μg PDNA only; an empty square indicates saline injection followed by electroporation; a solid triangle indicates injection of 50 μg PDNA followed by electroporation; a solid square indicates injection of 100 μg PDNA followed by electroporation.

FIG. 2 provides data confirming that non-coding sequences enhance the antitumor effect. (a)Tumor volumes and (b) tumor free animals after delivery of 100 μg plasmid DNA (VR1255) using electroporation on days 0, 3, and 7. Wherein, an empty circle indicates no treatment; a plus sign indicates injection of 100 μg PDNA only; and a solid square indicates injection of 100 μg PDNA followed by electroporation and n=6-7.

In an additional exemplary embodiment, melanomas were established in the flanks of C57B1/6 mice by injecting 1 million cultured B16 murine melanoma cells subcutaneously into each mouse. After a period of approximately 7 days, tumors had grown to a size of approximately 40 mm2. The experiment included 4 different treatment groups. These groups received no treatment, electric pulses alone, PUC18 injections alone and PUC18 injection followed by electrical treatment. Tumors were injected with 100 μg of PUC18 empty plasmid when appropriate. Electrical treatment was applied by a caliper electrode grasping the tumor. The caliper plates served as electrodes to deliver 10 rectangular direct current pulses that were 5 milliseconds in duration with strength of 800V/cm to the appropriate tumors. The treatment was applied to each group on the initial day of the experiment, day 0, and then on days 3 and 7 that followed. Tumor volume was calculated by the standard formula v=ab2π/6, where a is the longest diameter, and b is the next longest diameter perpendicular to a. Tumor volumes were determined at multiple time points starting on Day 0.

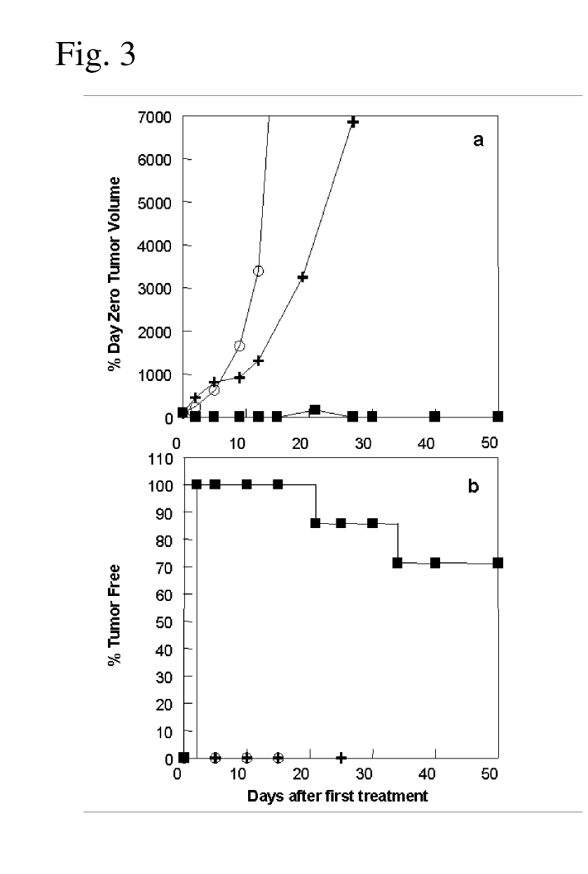

FIG. 3 illustrates the effect of three intratumor electroporation deliveries of plasmid DNA on tumor volume. FIG. 3a illustrates the tumor volumes and FIG. 3b illustrates the tumor free animals after delivery of PUC18 DNA with or without electroporation, in which an empty circle indicates no treatment; a plus sign indicates injection of 100 μg PDNA only; an empty diamond indicates saline followed by delivery of six 0.1 ms pulses at a field strength of 1500 V/cm; an empty square indicates saline injection followed ten 5 ms pulses at a field strength of 800 V/cm; a solid diamond indicates injection of 100 μg PDNA followed by delivery of six 0.1 ms pulses at a field strength of 1500 V/cm; a solid triangle indicates injection of 50 μg PDNA followed by ten 5 ms pulses at a field strength of 800 V/cm, a solid square indicates injection of 100 μg PDNA followed by electroporation protocol of ten 5 ms pulses at a field strength of 800 V/cm, and n=6-7 mice. The results obtained indicate that the group of mice treated with PUC18 plasmid followed by electrical treatment had dramatically reduced tumor volumes relative to the other treatment groups. In fact, the mean tumor volume of the animals that received PUC18 and electrical treatment was zero for all follow up days up to and including day 49 with exception of day 21. Eighty-five percent of the animals remained tumor free for the 49-day follow up period and 15 percent of animals had tumors that recurred on or after day 21 (FIG. 3b). These are striking antitumor effects when compared to the other treatment groups. These partial or no treatment groups all had mean tumor volumes that increased over time, and no animals were tumor free beyond day 0. These strong antitumor effects indicate that the combination of the energy driven delivery method was required to achieve deleterious effects using a nucleic acid sequence that did not contain any mammalian DNA sequences.

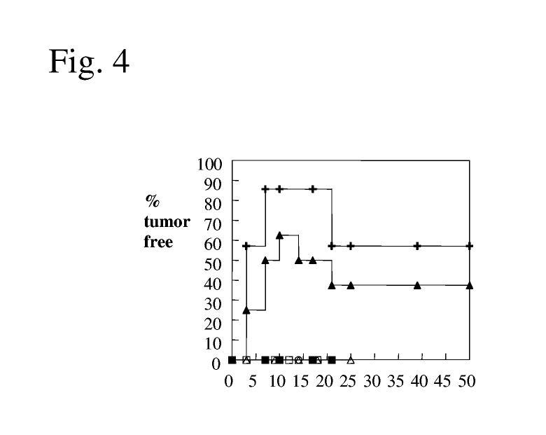

With reference to FIG. 4, in an additional exemplary embodiment, it is demonstrated that oligonucleotides only 20 bases long can elicit this anti-tumor effect in accordance with the present invention. Although the use of oligonucleotides only 20 bases in length may not elicit an effect to the astounding degree that plasmid DNA does, they are still show to be effective in eliciting an anti-tumor effect. In the exemplary embodiment as illustrated by FIG. 4, it is shown that electrically mediated delivery of CpG motif oligonucleotide, but not control oligonucleotide, induces tumor regression. The graph illustrates the % that are tumor free after delivery of oligonucleotides, with or without electroporation on days 0, 3, and 7 after growth of tumors to a 4 mm diameter, in which: an empty circle indicates no treatment; an empty square indicates injection with only control oligonucleotide (TCCATGAGCTTCCTGATGCT3′) ; a solid square indicates injection of 100 μg control oligonucleotide followed by ten 5 ms pulses at a field strength of 800 V/cm; an encircled parentheses indicates injection only of 100 μg CpG oligonucleotide (5′TCCATGACGTTCCTGATGCT3′); a solid triangle indicates injection of 100 μg CpG oligonucleotide followed by ten 5 ms pulses at a field strength of 800 V/cm; a plus sign indicates injection of 100 μg luciferase plasmid DNA followed by ten 5 ms pulses at a field strength of 800 V/cm.

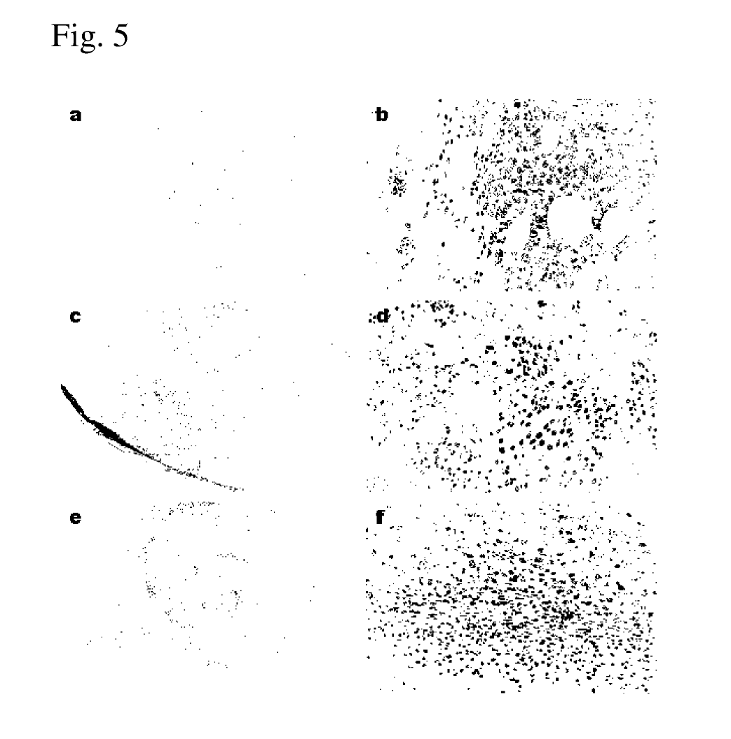

FIG. 5 shows a histological analysis of paraffin-embedded sections by hematoxylin and eosin (H&E) staining 24 hours after treatment. Specimens from mouse melanoma tumors were fixed in 10% neutral buffered formalin for 6 hrs. After fixation, the tissue samples were processed into paraffin blocks. Four micrometer-thick tissue sections were obtained from the paraffin blocks and stained with hematoxylin and eosin (H&E, Richard-Allan Scientific, Kalamazoo, Mich.) using standard histologic techniques. (a) untreated tumor, 40×; (b) untreated tumor, 250×; (c) injection of 100 μg VR1255 only, 40×; (d) injection of 100 μg VR1255 only, 250×; (e) saline injection followed by electroporation, 40×; (f) saline injection followed by electroporation, 250×. Histological analysis indicates that 80-100% of cells in treated tumors are apoptotic.

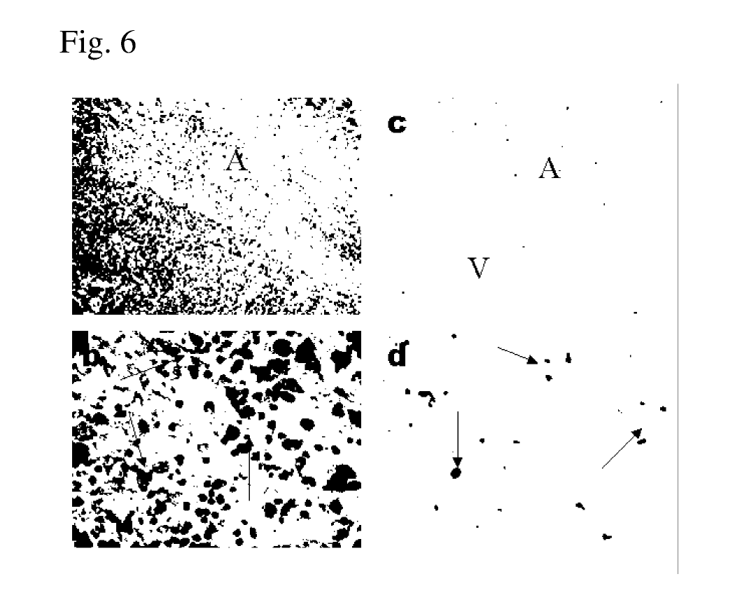

FIG. 6 shows a histological analysis of paraffin-embedded sections TUNEL staining 24 hours after delivery of 100 μg plasmid DNA (VR1255) using electroporation. Specimens from mouse melanoma tumors were bisected and half frozen at −70° C., and half was fixed in 10% neutral buffered formalin for 6 hrs. After fixation, the tissue samples were processed into paraffin blocks. Four micrometer-thick tissue sections were obtained from the paraffin blocks. Apoptosis was determined by TdT-mediated dUTP nick end labeling (TUNEL) using in situ cell death detection kit (Boehringer Mannheim). Frozen sections were prepared from the frozen tissues. The slides were fixed in paraformaldehyde (4% in PBS, pH 7.4). After rinsing with PBS and incubation in permeabilization solution, the tissue sections were cross reacted with TUNEL reaction mixture (for 60 min at 37° C. in a humidified chamber), with converter—alkaline phosphatase solution (for 30 min at 37° C. in a humidified chamber), and with alkaline phosphate substrate solution (Vector Laboratories, Burlington, Mass.) (for 5 to 10 min). The reactions were analyzed by light microscopy. (a) H&E, 100×; (b) H&E, 600×; (c) TUNEL, 100×; (d) TUNEL, 400×. A, apoptotic tumor cells; V, viable tumor cells, arrows indicate apoptotic cells (brown stained cells on the TUNEL assay).

Numerous other ways of practicing the invention described in this application are possible. These include, but are not limited to, the two components of the method which are the molecule(s) that are being transformed from nontherapeutic to therapeutic and the type of energy used for delivering the molecule(s). Each of these components are described below.

The nucleic acid molecule used for therapy generally includes one or more copies of a nucleic acid sequence; it can also include one or more copies each of two or more different nucleic acid sequences. Each nucleic acid sequence can be one or more nucleic acids long and composed of deoxyribonucleic acid (DNA), ribonucleic acid (RNA) derived from a mammalian, plant, fungal, viral, bacterial, or synthetic source. Nucleic acid sequences can be from any combination of these sources and may also include nucleic acids with sulfur, protein, or other backbones. The nucleic acid used for therapy may be in the form of a single strand, double strands, triplex strands composed of one or more DNA and one or more RNA strands, a DNA strand coupled to an RNA strand. Furthermore, nucleic acid may be defined for the purposes of this invention as any other molecule that may be a byproduct, contaminant, associated molecule, or other entity that results from the propagation, synthesis, handling, and/or purification process used to obtain the nucleic acids. In addition, the therapeutic effect may result in from the delivery of more than one type of nucleic acid or a combination of nucleic acid(s).

The nucleic acid in this invention may be a sequence that has no relevance to the body or organism that is being treated with the combination of energy source and nucleic acids. These irrelevant or non-coding nucleic acids may be of a form that is not from the organism being treated. For example, nucleic acids propagated in bacteria that contain no mammalian sequences that are used to obtain a therapeutic benefit in a mammal. Other examples to further exemplify this point may be but are not limited to: synthetic nucleic acids that code for no mammalian transcription or translation products used for therapy in a mammal; combined viral and bacterial sequences that have no mammalian sequences used for therapy in a mammal; and prokaryotic nucleic acid sequences that are used for therapy in a mammal. The nucleic acid sequence may also be of a form that is compatible with the host organism by does not code for a known transcription or translation product.

This invention may be practiced with different forms of energy that serve to transform the nontherapeutic nucleic acids into a therapeutic form. As indicated in the examples and associated figures, electricity can be used to produce this transformation by a mechanism that is assumed to at least partially include permeabilizing cell membranes to allow the nucleic acid access to the interior of cells. Energy in the form of sound waves which may be in the form of, but not limited to ultrasonic energy, could be used to transfer energy to the molecules and host system. Light is another form of energy that can be transferred to the nucleic acid and host. Laser light, for example, is one envisioned form of light energy. Finally, electromagnetic energy in the form of microwaves can also be used to apply energy to the system composed of the host and molecules of interest.

Furthermore, organic and inorganic chemical substances (chemicals) may facilitate the transformation of the nontherapeutic nucleic acids to a therapeutic form. For example, the addition of solubilized, emulsified, or suspended chemicals may facilitate energy transfer to the nucleic acids, host, or both. These chemicals may perform, for example, such functions as modifying electrical conductivity. They may also alter ultrasound, light, and microwave penetration.

The present invention is an It will be seen that the objects set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A method of eliciting an antitumor effect in vivo comprising the steps of:
identifying a treatment subject;
identifying at least one non-coding nucleic acid sequence, wherein the non-coding nucleic acid sequence is not associated with the expression of a gene of the treatment subject;
intratumorally introducing the at least one non-coding nucleic acid sequence to at least one tumor cell in the treatment subject; and
applying energy from an energy source to the at least one tumor cell, the application of the energy effective in eliciting an antitumor effect.

2. The method of claim 1 wherein the energy source is an electrical energy source.

3. The method of claim 1 wherein the step of applying energy from an energy source, further comprises making at least one cell in the at least one tumor permeable.

4. The method of claim 2 wherein the electrical energy source is an electrical source having a strength between 20 to 5,000 volts per centimeter.

5. The method of claim 2 wherein the electrical energy source is an electrical energy source comprising a plurality of electrical pulses.

6. The method of claim 1 wherein the step of introducing the at least one non-coding nucleic acid to at least one tumor cell in the treatment subject comprises injecting the nucleic acid into extracellular space coincident to the at least one tumor.

7. The method of claim 1 wherein the step of introducing the at least one non-coding nucleic acid to at least one tumor cell in the treatment subject comprises jet injecting the nucleic acid into extracellular space coincident to the at least one tumor.

8. The method of claim 1 further comprising the step of substantially simultaneously introducing a second nucleic acid sequence that codes for a therapeutic molecule.
